(19) United States
(12) Patent Application Publication
(10) Pub. No.: US 2024/0138813 A1
(43) Pub. Date: May 2, 2024

(54) POWER STRUCTURE AND ULTRASOUND PROBE

(71) Applicant: WUHAN UNITED IMAGING HEALTHCARE CO., LTD., Wuhan (CN)

(72) Inventors: Lei ZHANG, Wuhan (CN); Xianglong MA, Wuhan (CN); Kang SI, Shanghai (CN)

(73) Assignee: WUHAN UNITED IMAGING HEALTHCARE CO., LTD., Wuhan (CN)

(21) Appl. No.: 18/495,739

(22) Filed: Oct. 26, 2023

(30) Foreign Application Priority Data
Oct. 26, 2022 (CN) 202222833954.3
Dec. 28, 2022 (CN) 202223510208.7

(51) Int. Cl. A61B 8/00; G01S 7/52; G01S 15/89

(52) U.S. Cl. CPC A61B 8/4461; G01S 7/52079; G01S 15/8993

(57) ABSTRACT

One or more embodiments of the present disclosure may provide a power structure and an ultrasound probe. The power structure may include a driving assembly. The driving assembly may include a transmission device; the transmission device including a rotation component, a rope and an elastic component, and the rotation component including a driving wheel and/or a driven wheel. The driving wheel may be in a transmission connection to the driven wheel by the rope. The rope may be in an elastic connection to a body of the driving wheel and/or a body of the driven wheel through the elastic component. The rotation component may be provided with an accommodation cavity, and an extension channel in communication with the accommodation cavity. The elastic component may be accommodated in the accommodation cavity. One end of the rope elastically connected to the driving wheel and/or the driven wheel may be located in the accommodation cavity, and a portion of the rope may be located in the extension channel. The ultrasound probe may include a power structure and a transducer. The transducer may be configured to transmit and receive an ultrasound signal, and the transducer may be connected to the driven wheel of the power structure.

### CROSS-REFERENCE TO RELATED APPLICATION

This application claims priority of Chinese Patent Application No. 202222833954.3 filled on Oct. 26, 2022, and the Chinese application No. 202223510208.7 filed on Dec. 28, 2022, the contents of each of which are entirely incorporated herein by reference.

### TECHNICAL FIELD

The present disclosure relates to the technical field of a medical device, and in particular relates to a power structure and an ultrasound probe.

### BACKGROUND

An ultrasonic three-dimensional (3D) imaging is generally performed by a transducer in a probe transmitting and receiving ultrasonic waves intermittently in a process of oscillating or rotating to obtain two-dimensional (2D) images of different sections of a detected portion, and by a calculation process in combination with position information of the oscillation or rotation, a 3D stereo image is obtained. While a four-dimensional (4D) imaging requires an additional dimension of time based on 3D imaging. The 4D imaging is implemented by a reciprocal swinging or a multi-turn rotation to obtain the detection of 2D images of different sections, and then a real-time 3D stereo image is obtained by the calculation process. A power structure is utilized in the ultrasound probe to control an posture of a transducer, and a high degree of precision is required in a process of power transmission by the power structure.

Therefore, it is desirable to provide an improved power structure and ultrasonic probe with high accuracy.

### SUMMARY

One or more embodiments of the present disclosure provide a power structure including a driving assembly. The driving assembly may include a transmission device including a rotation component, a rope, and an elastic component. The rotation component may include a driving wheel and/or a driven wheel. The driving wheel may be transmissively connected to the driven wheel through the rope. The rope may be elastically connected to a body of the driving wheel through the elastic component. The rope may further be elastically connected a body of the driven wheel through the elastic component. An accommodation cavity and an extension channel communicates with the accommodation cavity may be disposed in the rotation component, the elastic component being accommodated in the accommodation cavity. One end of the rope elastically connected to the driving wheel and/or the driven wheel may be located in the accommodation cavity, and a portion of the rope being located in the extension channel.

One or more embodiments of the present disclosure provide an ultrasound probe. The ultrasound probe may include a power structure and a transducer. The transducer may be used for transmitting and receiving an ultrasound signal. The transducer may be connected to the driven wheel of the power structure.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating a structure of an exemplary transmission device according to some embodiments of the present disclosure;

FIG. 2 is a schematic diagram illustrating a structure of an exemplary driving wheel cooperating with a rope according to some embodiments of the present disclosure;

FIG. 3 is a schematic view illustrating a structure of an exemplary driving wheel in a sectional view state cooperating with a rope, according to some embodiments of the present disclosure;

FIG. 4 is a schematic diagram illustrating a sectional structure of an exemplary driving wheel according to some embodiments of the present disclosure;

FIG. 5 is a schematic diagram illustrating a mounting of a detection component according to some embodiments of the present disclosure;

FIGS. 6a-6c are schematic diagrams illustrating changes of relative positions of a sensor and a detection component during a movement of a driven wheel or a transducer base according to some embodiments of the present disclosure;

FIGS. 7a-7c are schematic diagrams illustrating changes of the relative positions of a sensor and a detector of a position detection device during a synchronous movement with a rotation component according to some embodiments of the present disclosure;

FIG. 8 is a schematic diagram illustrating a correspondence between a sensor output signal and a transducer unit rotation angle according to some embodiments of the present disclosure;

FIG. 9 is a schematic diagram illustrating a structure of a non-semicircular sector shaped detection component according to some embodiments of the present disclosure, and a schematic diagram illustrating a change of relative positions of a sensor and a detection component during a movement of a transducer unit according to some embodiments of the present disclosure;

FIG. 10 is a schematic diagram illustrating a detection component with a hollow structure according to some embodiments of the present disclosure;

FIG. 11 is a schematic diagram illustrating a structure of a plurality of detection regions according to some embodiments of the present disclosure;

FIG. 12 is a schematic diagram illustrating another structure of a plurality of detection regions according to some embodiments of the present disclosure;

FIG. 13 is a schematic diagram illustrating a structure of a power structure according to some embodiments of the present disclosure;

FIG. 14 is a schematic diagram illustrating a structure of a driving assembly according to some embodiments of the present disclosure;

FIG. 15 is a schematic diagram illustrating a position of a sensor mounted on a mounting base, and a schematic diagram illustrating a detection component mounted on an output shaft of a driving motor according to some embodiments of the present disclosure;

FIG. 16 is a schematic diagram illustrating a structure of an ultrasonic probe according to some embodiments of the present disclosure;

FIG. 17 is a schematic diagram illustrating a portion of structure of an ultrasonic probe in one perspective according to some embodiments of the present disclosure;

FIG. 18 is a schematic diagram illustrating a portion of structure of an ultrasonic probe in another perspective according to some embodiments of the present disclosure;

FIG. 19 is a schematic diagram illustrating a structure of a transducer cooperating with a driving wheel in one perspective according to some embodiments of the present disclosure;

FIG. 20 is a schematic diagram illustrating a structure of a transducer cooperating with a driven wheel in another perspective according to some embodiments of the present disclosure;

FIG. 21 is a schematic diagram illustrating a structure of a power structure in one perspective according to some embodiments of the present disclosure; and

FIG. 22 is a schematic diagram illustrating a structure of a power structure in another perspective according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios in accordance with these drawings without creative labor. The present disclosure may be applied to other similar scenarios based on these drawings without the expenditure of creative labor. Unless obviously obtained from the context or the context illustrates otherwise, the same numeral in the drawings refers to the same structure or operation.

As shown in the present disclosure and the claims, unless the context clearly suggests an exception, the words “a”, “an”, “one”, “a kind”, and/or “the” do not refer specifically to the singular, but may also include the plural. Generally, the terms “including” and “comprising” suggest only the inclusion of clearly identified operations and elements. In general, the terms “including” and “comprising” only suggest the inclusion of explicitly identified operations and elements that do not constitute an exclusive list, and the method or apparatus may also include other operations or elements.

An ultrasonic probe refers to a device that transmits and receives ultrasonic waves during an ultrasonic detection. The ultrasonic probe may generally be disposed with a transducer unit within a transducer for transmitting and receiving a signal. The transducer unit may be made to oscillate within a certain angle under a transmission of the driving device. When the ultrasound probe images a human tissue, the human tissue may be imaged with the help of the transducer unit that is able to oscillate within a certain angle within a desired angle, so as to obtain a three-dimensional (3D) image or a four-dimensional (4D) image of the human tissue.

The ultrasound probe may include ultrasound probes for body surface and ultrasound probes for internal use. For example, for the ultrasound probe for internal use, a rope transmission may often be used to transmit a long-distance rotational movement, so as to increase a scanning range of the ultrasound probe, thereby satisfying application scenarios of the ultrasound probe. As the rope is always stretched, when the driving device is activated, an end of the transducer may be impacted and a reliability of the device may be affected. Additionally, after a certain period of time, the rope may be worn out and slacken, which affects an transmission accuracy. In view of the foregoing, some embodiments of the present disclosure provide a power structure with a high transmission accuracy.

In some embodiments, the power structure may include a driving assembly.

In some embodiments, the driving assembly may include a transmission device, and the transmission device may include a rotation component. In some embodiments, the rotation component may achieve a power transmission by rotating, e.g., the rotation component may drive other structures to rotate by rotating itself to output a torque.

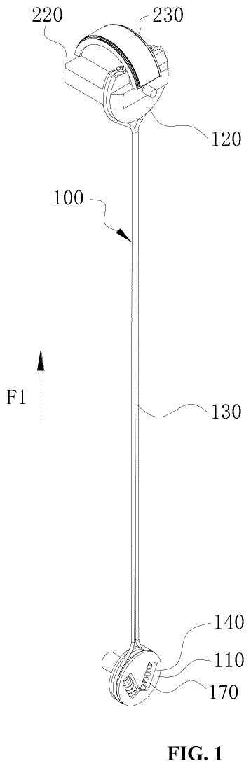

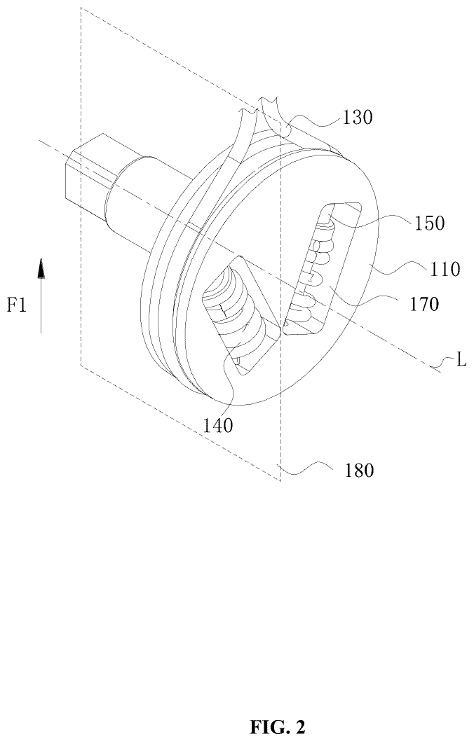

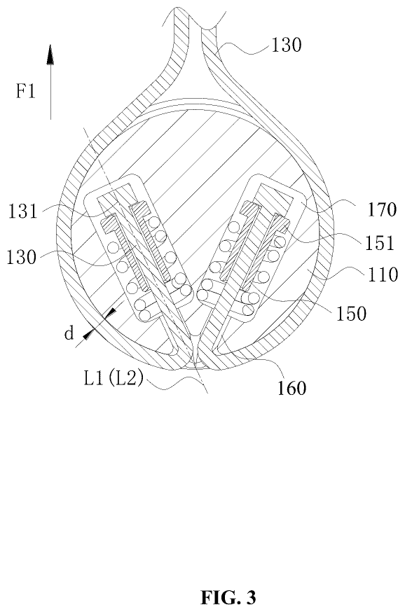

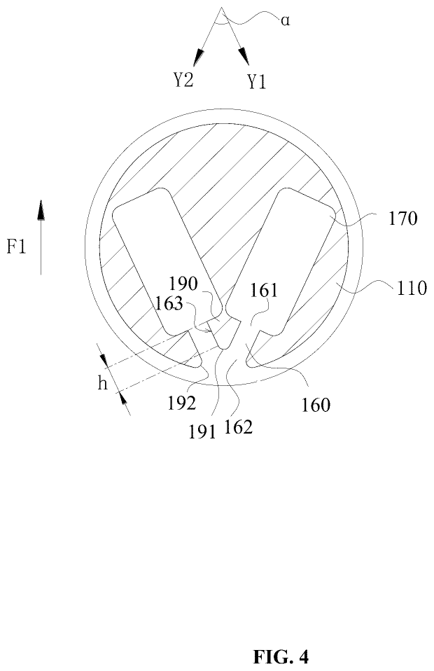

FIG. 1 is a schematic diagram illustrating a structure of an exemplary transmission device according to some embodiments of the present disclosure. FIG. 2 is a schematic diagram illustrating a structure of an exemplary driving wheel cooperating with a rope according to some embodiments of the present disclosure. FIG. 3 is a schematic view illustrating a structure of an exemplary driving wheel in a sectional view state cooperating with a rope according to some embodiments of the present disclosure. FIG. 4 a schematic diagram illustrating a sectional structure of an exemplary driving wheel according to some embodiments of the present disclosure.

In some embodiments, as shown in FIGS. 1-4, a driving assembly may include a transmission device 100.

The transmission device 100 may include a rotation component, a rope 130, and at least one elastic component 140. The rotation component may include a driving wheel 110 and/or a driven wheel 120. The driving wheel 110 may be connected to the driven wheel 120 by the rope 130. In some embodiments, the rope 130 may be elastically connected to a body of the driving wheel 110 through the elastic component 140. In some embodiments, the rope 130 may be elastically connected to the body of the driven wheel 120 through the elastic component 140.

In some embodiments, one end of the rope 130 may be elastically connected to the driving wheel 110 through the elastic component 140. In some embodiments, one end of the rope 130 may be elastically connected to the driven wheel 120 through the elastic component 140. In some embodiments, both ends of the rope 130 may be elastically connected to the driving wheel 110 and the driven wheel 120, respectively through the elastic component 140. In some embodiments, both ends of the rope 130 may be connected to the driving wheel 110 through the elastic component 140. In some embodiments, both ends of the rope 130 may be connected to the driven wheel 120 through the elastic component 140 respectively. For related contents on a specific mode in which the rope 130 is connected to the rotation component (the driving wheel, the driven wheel) through the elastic component 140, a count of the ropes 130, a mode in which the ropes 130 are disposed on the driving wheel and the driven wheel, please refer to the descriptions as follows.

The elastic component may be a spring. In some embodiments, one end of the elastic component may be connected to one end of the rope by bonding, welding, bolting, etc. In other embodiments, the elastic component, and the rope 130 may be an integrated structure.

In some embodiments, a driving force may be transmitted to the driven wheel 120 through the driving wheel 110 and the rope 130. A transmission of the rope 130 has a good flexibility, making a drive process smoother and avoiding a noise, a vibration, and an impact.

In some embodiments, there may be one or more ropes 130. The rope 130 and the driving wheel 110, as well as the rope 130 and the driven wheel 120 may be connected through a direct fixation and an indirect fixation through the elastic component 140. For example, when there is one rope 130, the rope 130 may be disposed around the driven wheel 120 for one turn, and a fixing point may be disposed on the driven wheel 120, thereby preventing the rope 130 from sliding relative to the driven wheel 120. Both ends of the rope 130 may be connected to the driving wheel 110. At this time, at least one of end of the rope 130 may be elastically connected to the driving wheel 110 through the elastic component 140. For another example, when there are two ropes 130, each rope 130 may be connected between the driving wheel 110 and the driven wheel 120. That is, one end of each of the two ropes 130 may be connected to the driving wheel 110, and the other end of each of the two ropes 130 may be connected to the driven wheel 120. When the driving wheel 110 is driven, the rope 130 connected to the driving wheel 110 may be driven to act, which in turn drives the driven wheel 120 connected to the rope 130 to move, thereby realizing the transmission connection between the driving wheel 110 and the driven wheel 120.

In some embodiments, it may be understood that when the rope 130 is directly fixed to the driving wheel 110 and the driven wheel 120, a count of fixing points as well as arrangement positions of the fixing points may be determined according to the count of the ropes 130 as well as a specific mounting mode. In some embodiments, the disposal of the count of the rope 130 may be determined according to the transmission accuracy required and structure and arrangement of the driving wheel 110 and the driven wheel 120.

Exemplarily, one end of each of the two ropes 130 may be fixedly connected to the driven wheel 120, as shown in FIG. 1. Two fixing points on the driven wheel 120 may be located on either side of a plane that passes a rotation axis of the driving wheel 110 and a rotation axis of the driven wheel 120. In some embodiments, the two fixing points may also overlap at one point, which may be disposed according to a specific use situation. In some embodiments, whether there is one rope 130, or two ropes 130, as long as a portion of the rope 130 disposed between the driving wheel 110 and the driven wheel 120 is capable of being disposed on two sides of the plane that passes the rotation axis of the driving wheel 110 and the rotation axis of the driven wheel 120 to avoid tangling of the rope 130 in the portion disposed between the driving wheel 110 and the driven wheel 120, the embodiments of the present disclosure makes no limitation on the count of the rope 130. While the tangling of the rope 130 during the rotations of the driving wheel 110 and the driven wheel 120 is avoided, a non-linear change in the rotations is avoided as well, so as to avoid affecting the transmission accuracy.

It is to be noted that when the two ropes 130 are disposed, accordingly, at least one end of at least one of the two ropes 130 may be connected to the driving wheel 110 or the driven wheel 120 through the elastic component 140, i.e., according to a dispose position of the elastic component 140, a connection mode of the two ropes 130 to the driving wheel 110 or the driven wheel 120 may include, but may not be limited to, the following situations: (1) one end of one of the two ropes 130 may be connected to the driving wheel 110 through the elastic component 140; (2) one end of one of the two ropes 130 may be connected to the driven wheel 120 through the elastic component 140; (3) one end of one of the two ropes 130 may be connected to the driving wheel 110, and one end of the other of the two ropes 130 may be connected to the driving wheel 110 through the elastic component 140; and (4) one end of one of the two ropes 130 may be connected to the driven wheel 120 through the elastic component 140, and one end of the other of the two ropes 130 may be connected to the driven wheel 120 through the elastic component 140; and (5) one end of one of the two ropes 130 may be connected to the driving wheel 110 through the elastic component 140, and one end of the other of the two ropes 130 may be connected to the driven wheel 120 through the elastic component 140. In some embodiments, as shown in FIG. 2, one end of each of the two ropes 130 may be connected to the driving wheel 110 through the elastic component 140. The rope 130 may be disposed according to actual use, and the embodiments of the present disclosure do not make specific limit on this.

It should be noted that the body of the wheel refers to the driving wheel 110 or the driven wheel 120 which is connected to the rope 130 through the elastic component 140. That is, when the rope 130 is connected to the driving wheel 110 through the elastic component 140, the driving wheel 110 may be the body of wheel referred to; when the rope 130 is connected to the driven wheel 120 through the elastic component 140, the driven wheel 120 may be the body of wheel referred to; and when the rope 130 is connected to the driving wheel 110 and the driven wheel 120 respectively through the elastic component 140, the driving wheel 110 and the driven wheel 120 may both be the body of wheel referred to.

In some embodiments, the ropes are connected to the corresponding wheel bodies by elastic components, alleviating the problem of affecting a transmission accuracy and reliability due to the uncontrollable state of the ropes, and improving the transmission accuracy and reliability.

In some embodiments, as shown in FIGS. 2, 3, and 4, there may be an accommodation space within the rotation component that at least accommodates the elastic component 140. In some embodiments, the accommodation space for accommodating the elastic component 140 may be provided within the body of wheel of the driving wheel 110. In some embodiments, the body of the wheel of the driven wheel 120 may be provided with the accommodation space within the body of wheel that accommodates the elastic component 140. In some embodiments, both the driving wheel 110 and the driven wheel 120 may be provided with the accommodation space within the body of wheel that accommodates the elastic component 140.

In some embodiments, the end of the rope 130 may be accommodated in the accommodation space with the elastic component 140.

In some embodiments, by accommodating the elastic component 140 within the accommodation space, an interference between the elastic component 140 and a housing of the ultrasound probe is avoided, which in turn avoids a limited use of the ultrasound probe within the human body. In addition, the elastic component 140 may subject to a longitudinal bending or a transverse bending during the transmission process, and the bending of the elastic component 140 may generate a higher stress locally, which leads to a fracture in a severe situation, thereby affecting the accuracy and reliability of the transmission. The elastic component 140 may avoid the bending by being stored in the storage space, which improves a stress situation of the elastic component 140, and thus improves the accuracy and reliability of the transmission.

In some embodiments, as shown in FIGS. 2, 3, and 4, the accommodation space may include an accommodation cavity 170, and an extension channel 160 that is in communication with the accommodation cavity 170. The elastic component 140 may be accommodated within the accommodation cavity 170. The end of the rope 130 elastically connected to the body of wheel may be located within the accommodation cavity 170, and a portion of the rope 130 may be located within the extension channel 160.

The accommodation cavity 170 may be used to accommodate the elastic component 140 and/or one end of the rope 130. In some embodiments, as shown in FIG. 2, the accommodation cavity 170 may be opened on an end face of the driving wheel 110. In some embodiments, the accommodation cavity 170 may also be opened inside the driving wheel 110. In some embodiments, as shown in FIG. 2, the accommodation cavity 170 may be a rectangular slot opened on the end face of the driving wheel 110.

It may be understood that, as the accommodation cavity 170 is disposed on the body of wheel, that is, the elastic component 140 is disposed inside the driving wheel 110 or the driven wheel 120. As a result, an internal space of the driving wheel 110 or the driven wheel 120 may be utilized even more effectively.

The extension channel 160 may act as a limitation on the end of the rope 130 and may be used to guide the rope 130 into the accommodation cavity 170. In some embodiments, the extension channel 160 may be on an one-to-one communication with the accommodation cavity 170. In some embodiments, a position, and a count of the accommodation cavity 170 may correspond to a position and a count of the elastic component 140, and a position and a count of the extension channel 160 may correspond to a position and a count of the accommodation cavity 170. For example, two elastic components 140 may be provided within the body of wheel, whether there is one rope 130 or two ropes 130 provided, two ends of the rope 130 may be elastically connected to the body of wheel. When there is one rope 130, both ends of the rope 130 connected to the driving wheel 110 may be elastically connected to the driving wheel 110. When there are two ropes 130, when one of the two ends of the two ropes 130 elastically connected to the wheel body, the body of the wheel may be provided with two accommodation cavities 170 and two extension channels 160, with each accommodation cavity 170 provided with one elastic component 140, and each of the accommodation cavities 170 may be connected to one of the extension channels 160. The end of the rope 130 elastically connected to the body of the wheel may extend into the accommodation cavity 170 through the extension channel 160, and with the help of the elastic component 140 in the accommodation cavity 170, the rope 130 may be connected to the corresponding body of the wheel.

The elastic component 140 may be used to provide an elasticity acting on a corresponding end of the corresponding rope 130. The elasticity may be capable of making the corresponding end of the corresponding rope 130 to tend to move away from the extension channel 160, thereby ensuring that the rope 130, after a period of time of use, still remains taut. Due to the elastic force, the rope 130 may be made to extend from the accommodation cavity 170 to the extension channel 160 and then extend out from the extension channel 160.

It may be noted that the elasticity provided by the elastic component 140 may be either a tensile force generated by the elastic component 140 or a compressive force generated by the elastic component 140. For example, when one end of the elastic component 140 is connected to an inner wall of the accommodation cavity 170 and the other end is connected to the corresponding end of the corresponding rope 130, and the rope 130 is in a tensioned state, the elasticity provided by the elastic component 140 may be the tensile force generated by the elastic component 140. For the situation in which the elasticity provided by the elastic component 140 is the compressive force generated by the elastic component 140, reference may be made to some of the later embodiments, which is not repeated herein. Thereby, a structural form in which the elastic component 140 is used in conjunction with the corresponding end of the corresponding rope 130 may be correspondingly disposed according to the actual needs of use, and the embodiments of the present disclosure do not impose any specific limitations thereon.

In some embodiments, the elastic component 140 may be accommodated within the accommodation cavity 170, i.e., the elastic component 140 may be confined within the accommodation cavity 170, and the elastic component 140 may not protrude into the extension channel 160. When the rope 130 extends from the accommodation cavity 170 through the extension channel 160, due to a presence of the extension channel 160, the stress situation of the elastic component 140 is improved, so as to prevent the elastic component 140 from bending, which in turn allows the rope 130 to perform the transmission in the tensioned state.

In some embodiments, by disposing the elastic component 140 in the corresponding body of wheel, and disposing the accommodation cavity 170 adapted to the elastic component 140, the extension channel 160 adapted to the accommodation cavity 170, the elastic component 140 may provide the elasticity acting on the rope 130. In this way, the stress situation of the elastic component 140 is improved, so as to avoid the bending of the elastic component 140, which in turn allows the rope 130 to perform transmission under the tensioned state. As a result, the transmission accuracy may not be affected due to a wear of the rope 130 and the bending of the elastic component, and the transmission accuracy is improved.

In some embodiments, an elasticity direction of the elastic component 140 may be parallel to an extension direction of the extension channel 160. In some embodiments, the elastic component 140 may be disposed as a spring, and an axis of the spring, which is in the elasticity direction of the spring, may be parallel to an axis of the extension channel 160.

In some embodiments, as shown in FIG. 3, the axis of the elastic component 140 may be a first axis L1, and the axis of the extension channel 160 may be a second axis L2, with the first axis L1 and the second axis L2 being coextensive (i.e., on the same straight line) with each other. That is, the elasticity generated by the elastic component 140 may be generated along the axis of the extension channel 160.

In some embodiments, by disposing the elasticity direction of the elastic component 140 parallel to the extension direction of the extension channel 160, the situation of bending of the elastic component 140 is better improved, thereby improving the accuracy and reliability of the transmission even further.

In some embodiments, as shown in FIGS. 2-4, the extension channel 160 may include a linear extension section 163. In some embodiments, a ratio of a length h of the linear extension section 163 to a diameter d of the rope 130 within the straight line extension section 163 may be greater than 1.5. In this way, the rope 130 may be well guided through the extension channel 160.

In some embodiments, by providing the extension channel 160 with the straight line extension section 163, it may be possible to make the elasticity direction of the elastic component 140 coincide as much as possible with a protrusion direction of the rope 130 disposed within the extension channel 160, and thus the elasticity direction of the elastic component 140 may be aligned with the axial direction of the elastic component 140 as much as possible to avoid a bending deformation of the elastic component 140, thereby improving a problem of bending deformation of the elastic component 140.

In some embodiments, as shown in FIGS. 2-4, the transmission device 100 may include two elastic components 140, with two accommodation cavities provided within the rotation component (e.g., the driving wheel 110 or the driven wheel 120), and the two elastic components 140 may be located in the two accommodation cavities; the two elastic components 140 may be disposed on both sides of a radial reference surface 180 of the rotation component. The radial reference surface 180 may be a plane passing through a central axis L of the driving wheel 110. In some embodiments, an axis of the center of rotation of the rotation component may be located on the radial reference plane 180.

In some embodiments, both elastic components 140 may be disposed within the driving wheel 110, or both elastic components 140 may be disposed within the driven wheel 120. In some embodiments, one of the two elastic components 140 may be disposed within the driving wheel 110 and the other elastic component 140 may be disposed within the driven wheel 120. In some embodiments, both elastic components 140 may be disposed within the driving wheel 110, or both elastic components 140 may be disposed within the driven wheel 120, which further improves a space utilization.

In some embodiments, the two elastic components 140 may respectively be disposed on two sides of the radial reference surface 180 of the driving wheel 110, as shown in FIGS. 2-4. Further, the two elastic components 140 may be disposed symmetrically on both sides of the radial reference surface 180 to improve the accuracy and stability of the transmission through the symmetrical structure. In some embodiments, the two elastic components 140 may also be disposed on both sides of the radial reference surface 180 in an asymmetric manner. The dispose of the elastic components 140 may be chosen according to actual use situation, and the embodiments of the present disclosure do not make specific limits herein.

In some embodiments, the extension directions of the extension channels 160 corresponding to the two elastic components 140 may be disposed at an angle, as shown in FIGS. 3 and 4. The angular dispose of the extension directions of the extension channels 160 corresponding to the two elastic components 140 may be understood as follows: the extension directions of the extension channels 160 corresponding to the two elastic components 140 may not be parallel to each other. In some embodiments, as shown in FIG. 4, the extension directions of the two extension channels 160 may respectively be a first extension direction Y1 and a second extension direction Y2. The first extension direction Y1 and the second extension direction Y2 may form an angle α. In some embodiments, the angle α may be an acute angle, which may be 20°, 30°, 40°, 50°, 60°, etc.

In some embodiments, as shown in FIG. 4, the extension channel 160 may have a first end 161 that is in communication with the corresponding accommodation cavity 170 and a second end 162 that is away from the corresponding accommodation cavity 170. In some embodiments, a distance between the first ends 161 of the two extension channels 160 may be greater than a distance between the second ends 162 of the two extension channels 160.

In some embodiments, the first ends 161 of the two extension channels 160 may extend at an angle away from each other, and the second ends 162 of the two extension channels 160 may be disposed close to each other. In this way, the two ropes 130 may be disposed close to each other at a position where the driving wheel 110 is protruded to obtain a great transmission ratio, thereby improving the transmission accuracy even further.

In some embodiments, as shown in FIG. 4, the second ends 162 of the two extension channels 160 may be in communication with each other, i.e., the distance between the second ends 162 of the two extension channels 160 may be zero, which allows the two ropes 130 extending to the driving wheel 110 closer together, so as to further increase the transmission ratio.

In some embodiments, a separation portion 190 may be formed between the two extension channels 160. An end portion of the separation portion 190 includes a first arc section 191, the first arc section 191 being disposed on a side of the separation portion 190 toward a connection position between the second ends 162 of the two extension channels 160. In some embodiments, by disposing the first arc section 191 on the separation portion 190, and by disposing the separation portion 190, a separation between the two ropes 130 may be generated to avoid a friction between the two ropes 130 during the transmission. By disposing a sharp portion of the separation portion 190 as the first arc section 191, it is possible to prevent the sharp portion of the separation portion 190 from damaging the rope 130 during the movement of the rope 130, and at the same time, it is possible to prevent the rope 130 from wearing out the separation portion 190 during the movement of the rope 130.

In some embodiments, as shown in FIG. 4, there may be a second arc section 192 between the second end 162 of the extension channel 160 and a circumference of a corresponding body of the wheel. The body of the wheel corresponding to the second end 162 of the extension channel 160 refers to a body of the wheel with the driving wheel 110 or the driven wheel 120 of the extension channel 160 disposed inside.

In some embodiments, taking the body of the wheel being the driving wheel 110 as an example, the second arc section 192 may be respectively formed between the second end 162 of each of the two extension channels 160 and the circumference of the driving wheel 110. In this way, the wear generated between the two ropes 130 and the driving wheel 110 during the transmission may be reduced. In some embodiments, the two second arc sections 192 may be arranged symmetrically on both sides of the radial reference surface 180.

In some embodiments, the surface of the second arc section 192 may be processed, for example, sprayed, polished, etc., to obtain a smoother surface. In this way, the friction generated between the rope 130 and the driving wheel 110 during the transmission process mat be further reduced to a certain extent.

In some embodiments, a roller (not shown in the drawings) may be disposed between the second end 162 of the extension channel 160 and the circumference of the corresponding rotation component (e.g., the body of the wheel being the driving wheel 110). The rope 130 may be disposed around the roller. The rope 130 and the roller may form a rolling connection to reduce the wear on the rope by the body of the wheel as the rope 130 moves. The connection between the rope 130 and the roller may also reduce a contact area of the rope 130 with the rotation component, thereby reducing the friction and wear between the rope 130 and the rotation component.

In some embodiments, as shown in FIG. 3, the power structure may further include a guiding component 150 disposed within the accommodation cavity 170, the guiding component 150 may guide the movement of the elastic component 140 along the elasticity direction of the elastic component 140. By disposing the guiding component 150, the bending deformation of the elastic component 140 is avoided.

In some embodiments, the elastic component 140 may be disposed outside of the guiding component 150, and the guiding component 150 may be disposed outside of a corresponding end of the rope 130 that extends into the accommodation cavity 170, and may be capable of restricting the corresponding end of the rope 130 to lie within the accommodation cavity 170. The rope 130 may pass through a central hole of the guiding component 150 and secured, and the guiding component 150 may prevent the elastic component 140 from bending.

In some embodiments, as shown in FIGS. 2, 3, and 4, a first limiting portion 151 may be provided at an end of the guiding component 150, and a second limiting portion 131 may be provided at the end of the rope 130 that extends into the accommodation cavity 170. The guiding component 150 may abut against the elastic component 140 with the help of the first limiting portion 151, and the rope 130 may be limited outside the guiding component 150 with the help of the second limiting portion 131. That is to say, the rope 130 may pass through the central hole of the guiding component 150 and may be limited by the second limiting portion 131. The guiding component 150 may pass through the center of the elastic component 140 and may be disposed together in the accommodation cavity 170 of the driving wheel 110. The rope 130 may pass from the accommodation cavity 170 into the extension channel 160, and then extend out from the extension channel 160, and wrap around the circumference of the driving wheel 110. The end of the elastic component 140 close to the extension channel P may be limited by the accommodation cavity 170, and the other end of the elastic component 140 may be compressed due to the pressure exerted on the elastic component 140 by a preload force of the rope 130 when the rope 130 is assembled. When compressed, the elastic component 140 may produce an opposite pulling force on the rope 130, making the rope 130 to be in a tense state thereby ensuring the tension of the rope transmission. In this way, it is possible to realize a more effective fixation of the rope 130, avoiding falling off during the transmission. In some embodiments, other configurations may be used to allow one end of the rope 130 to be limited within the accommodation cavity 170, and the guiding component 150 may guide the elastic component 140.

It may be appreciated that a length of the guiding component 150 is not greater than a length of the elastic component 140 so that the elasticity generated by the elastic component 140 may be effectively utilized.

In some embodiments, when one end of the elastic component 140 is connected to an inner wall of the accommodation cavity 170 and the other end is connected to a corresponding end of the corresponding rope 130, the elastic component 140 may be disposed outside of the guiding component 150 or the guiding component 150 may be disposed outside the elastic component 140, so that the guiding component 150 may guide the elastic component 140. In this way, the guiding component 150 may be flexibly disposed according to an arrangement between the elastic component 140 and the corresponding end of the corresponding rope 130, as well as usage requirements, as long as the guiding component 150 is able to guide the elastic component 140.

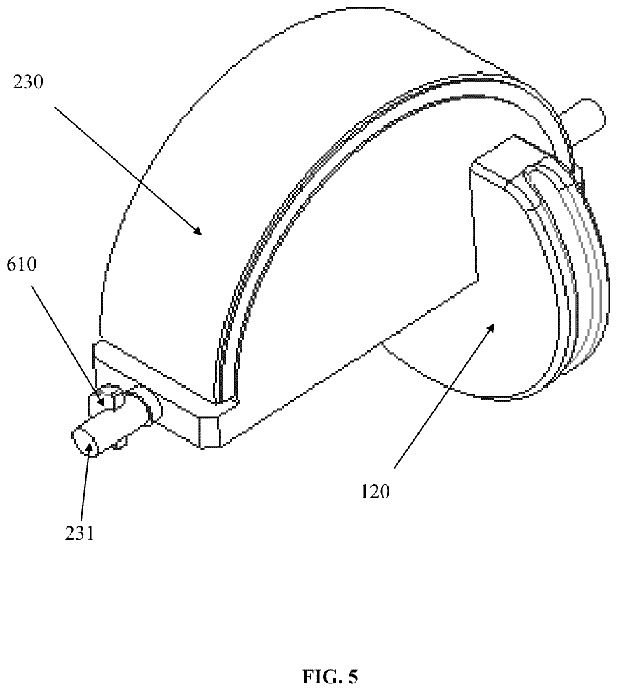

FIG. 5 is a schematic diagram illustrating a mounting of a detection component according to some embodiments of the present disclosure.

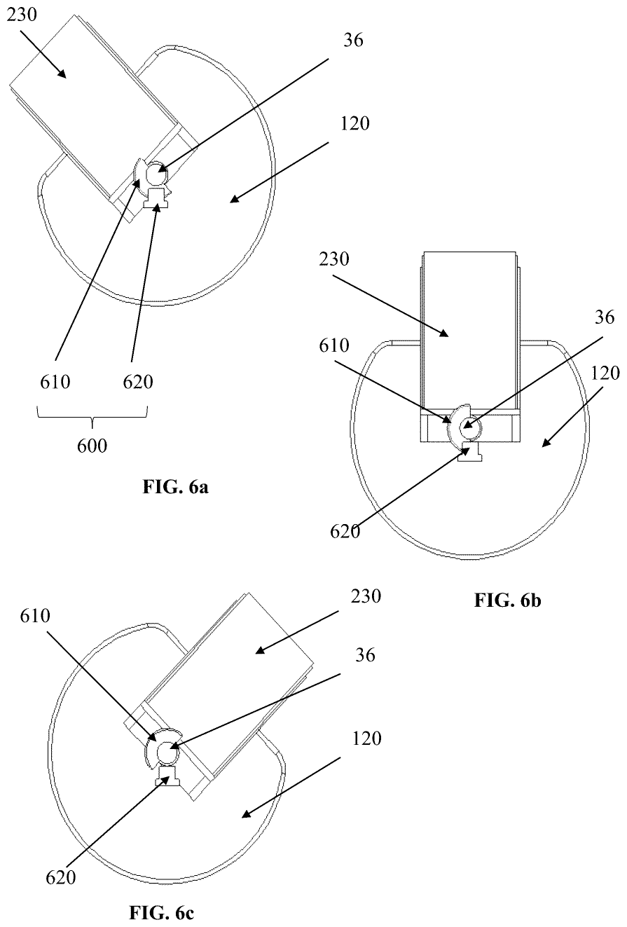

In some embodiments, as shown in FIG. 5, a power structure may further include a position detection device 600 (see FIG. 6a). The position detection device 600 may be used to monitor, for example, a position, a moving direction, and a state of a transducer unit 230. The position detection device 600 may be disposed on a rotation component.

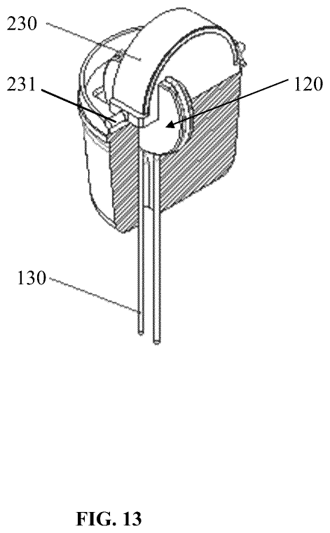

In some embodiments, the position detection device 600 may include a detection component 610 and a sensor 620. In some embodiments, the detection component may be disposed on the rotation component. In some embodiments, the detection component 610 may be disposed on the driven wheel 120 or a transducer base 220. In some embodiments, a transducer 200 may include the transducer unit 230 and the transducer base 220, and the transducer unit 230 may be mounted on the transducer base 220. The transducer unit 230 may be connected to the driven wheel 120 through the transducer base 220. For detailed contents on the transducer base 220, please refer to FIG. 13 and the related descriptions. In some embodiments, the driven wheel 120 may drive the transducer base 220 to rotate synchronously, thereby driving the detection component 610 to rotate synchronously. In some embodiments, the detection component 610 may be disposed on the rotation axis 231, the driven wheel 120 may drive the rotation axis 231 to rotate synchronously. In some embodiments, the rotation axis 231 may be configured to connect structures like a housing of an ultrasound probe, etc., and the rotation axis 231 may not move synchronously with the transducer unit 230. The detection component 610 may be disposed on other structures being able to move synchronously with the transducer unit 230. For example, the detection component 610 may be disposed on the transducer unit 230. For another example, the detection component 610 may be disposed on the driving wheel 110. For another example, the detection component 610 may be disposed on an output axis of a driving motor that drives the driving wheel 110 and the driven wheel 120 to rotate. The detection component 610 may be driven by the rotation component to move in synchronization with the rotation component, thereby synchronizing the movement of the detection component 610 and the transducer unit 230.

In some embodiments, the detection component 610 may include a first detection edge 611, a second detection edge 612, and a detection portion. The detection component may be disposed between the first detection edge 611 and the second detection edge 612.

In some embodiments, the sensor 620 may be configured to receive a detection signal, and the detection signal may be transmitted toward the detection component 610. In some embodiments, the sensor 620 may include a transmitting component and a receiving component. The transmitting component may be configured to transmit the detection signal, and the receiving component may be configured to receive the detection signal. When the transmitting component and the receiving component are disposed on the same side of the detection component 610, the receiving component may receive the detection signal reflected by the detection component 610. When the transmitting component and the receiving component are respectively disposed on both sides of the detection component 610, the receiving component may receive the detection signal transmitted by the transmitting component.

In some embodiments, a device for transmitting the detection signal of the position detection device 600 (e.g., the sensor 620) may be disposed opposite to the detection component 610, and may transmit the detection signal to a face where the detection component 610 is located. In some embodiments, the position of the sensor 620 may be fixed. In some embodiments, the opposite arrangement of the sensor 620 and the detection component 610 refers to an opposite positional relationship in which the sensor 620 is able to transmit the detection signal to the face where the detection component 610 is located. In some embodiments, the transmission of the detection signal may be perpendicular to the face where the detection component 610 is located. It may be understood that the detection signal perpendicular to the face where the detection component 610 is located is meant to be an approximately perpendicular (ideally it is absolutely perpendicular), which allows a slight deviation in an actual manufacturing process.

In some embodiments, during the synchronized movement of the detection component 610 with the rotation component (e.g., the driven wheel 120 or the transducer base 220), a first signal may be output by the sensor 620 when the detection signal is transmitted to the detection component 610, and a second signal may be output by the sensor 620 when the detection signal is not transmitted to the detection component 610. The first signal and the second signal may be different. The first signal and the second signal may be electrical signals. The synchronized movement of the detection component 610 and the driven wheel 120 or the transducer base 220 is in consistent with an opposite mounting plan of the two in the movement mode, and may both be the plan described in the embodiments of the present disclosure. For example, the detection component 610 may be fixedly mounted on the driven wheel 120, and during the rotation of the driven wheel 120, the detection component 610 may rotate synchronously with the driven wheel 120. For another example, the detection component 610 may be mounted on a portion of a driving component of the transducer base 220. While the transducer base 220 is oscillating in one direction, the sensor 620 may detect at least one transition of the first signal and the second signal, and transitional a position of the first signal and the second signal may be the position of the first detection edge 611 or the position of the second detection edge 612. As the detection component 610 and the transducer base 220 perform the synchronized movement, when the position of the first detection edge 611 or the second detection edge 612 is detected, the position of the first base 220 may also be uniquely determined. The position of the transducer base 220 and the position of the first detection edge 611 or the second detection edge 612 may correspond to each other.

In some embodiments, a detection component 610 with a constant position relative to the rotation component may be disposed on the rotation component.

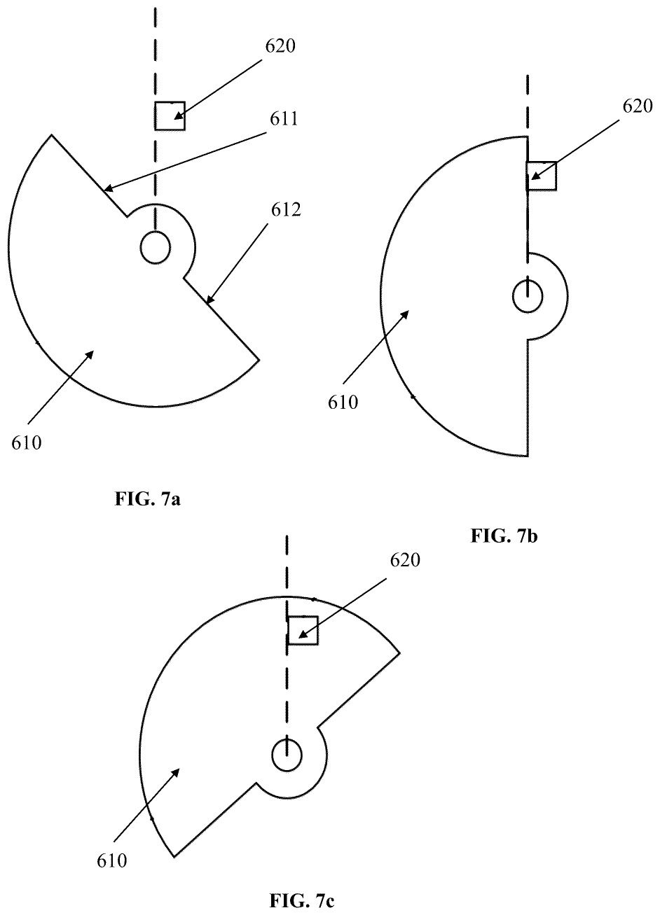

FIGS. 6a-6c are schematic diagrams illustrating changes of relative positions of a sensor and a detection component during a movement of a driven wheel or a transducer base according to some embodiments of the present disclosure. FIGS. 7a-7c are schematic diagrams illustrating changes of the relative positions of a sensor and a detector of a position detection device during a synchronous movement with a rotation component according to some embodiments of the present disclosure.

In some embodiments, as shown in FIGS. 6a-6c and FIGS. 7a-7c, the sensor 620 may be disposed perpendicular to a face on which the detection component 610 is located. Whether or not a detection signal transmitted by the sensor 620 is transmitted on the detection component 610 may produce different signals output by the sensor 620. The variation of the signal may indicate an oscillation of the driven wheel 120 or the transducer base 220. During the oscillation of the detection component 610, the first detection edge 611 or the second detection edge 612 of the detection component 610 may cut through the detection signal, and at this time, there may be a transition point between the first signal and the second signal. According to the transition point, the position of the first detection edge 611 or the second detection edge 612 of the detection component 610 may be determined, and the position of the detection component 610 may correspond to the position of the driven wheel 120 or the transducer base 220, so that the position of the driven wheel 120 or the transducer base 220 may be directly determined. Based on a characteristic that the signal output by the sensor 620 may transit at a moment of the first detection edge 611 or the second detection edge 612 of the detection component 610 cutting through the sensor when the detection component 610 oscillates synchronously with the driven wheel 120 or the transducer base 220, the position of the driven wheel 120 or the transducer base 220 may be determined more accurate and faster.

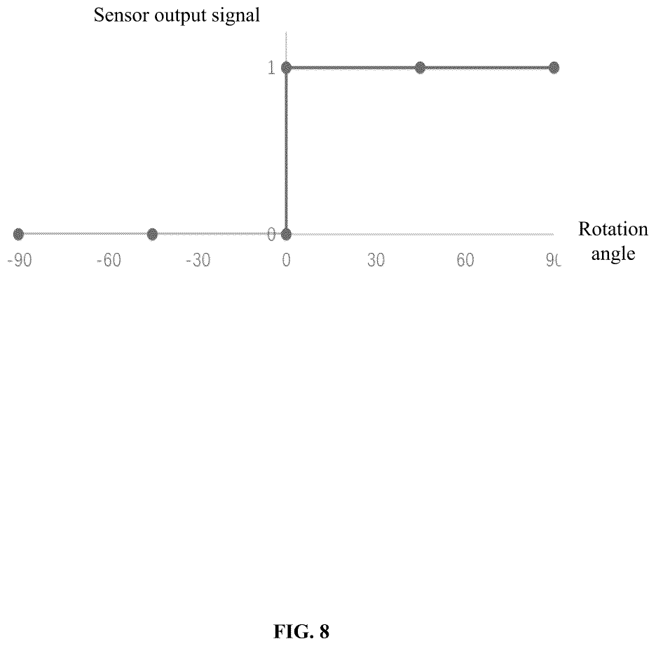

For example, the driven wheel 120 or the transducer base 220 may oscillate from left to right. FIGS. 6a-6c present several intermediate states during the oscillation. FIG. 8 is a schematic diagram illustrating a correspondence between a sensor output signal and a transducer unit rotation angle according to some embodiments of the present disclosure. During an oscillation of the driven wheel 120 or the transducer base 220, the detection component 610 may oscillate accordingly, while the sensor 620 is still. A relative position between the sensor 620 and the detection component 610 changes, and an output of the sensor 620 changes. When the driven wheel 120 or the transducer base 220 is in the state shown in FIG. 6a, the detection component 610 may shade the sensor 620, and the sensor 620 may show no signal received (e.g., supposing the situation being low level 0, the level of the output signal of the sensor 620 is related to a type of the sensor 620). When the driven wheel 120 or the transducer base 220 is in the state of FIG. 6c, the detection component 610 may not shade the sensor 620, and the sensor 620 may show a signal received (supposing the situation being high level 1), while FIG. 6b shows that the driven wheel 120 or the transducer base 220 is at a critical position in the middle. At this moment, an output pulse of the sensor 620 undergoes a transition as shown in FIG. 8.

In some embodiments, as shown in FIG. 6b, the transition position of the output signal of the sensor 620 corresponds to a middle position of the driven wheel 120 or the transducer base 220, and based on this correspondence, the middle position of the driven wheel 120 or the transducer base 220 may be determined quickly. In some embodiments, according to the needs in different scenarios, the transition moment as well as the positional relationship between the sensor 620 and the detection component 610 may be determined as the relative position state shown in FIG. 6b. The position of the driven wheel 120 or the transducer base 220 at the transition moment may be used as a set initial position to determine a specific position of the driven wheel 120 or the transducer base 220 at each moment. The selection of the set initial position may be implemented by changing the relative positional relationship between the detection component 610 and the driven wheel 120 or the detection component 610 and the transducer base 220 at the transition moment of the output signal of the sensor 620.

In some embodiments, the first detection edge 611 and the second detection edge 612 of the detection component 610 may both be perpendicular to a rotation direction of the rotation component. The first detection edge 611 and the second detection edge 612 may be perpendicular to the face on which the circumference formed by the rotation of the rotation component is located, and the first detection edge 611 and the second detection edge 612 may be collinear with any of a circumferential radius. There may be a circumcenter angle between the first detection edge 611 and the second detection edge 612, which may be less than or equal to 180°, such as 30°, 40°, 60°, 90°, 120°, 160°, 180°, etc.

During the oscillation of the detection component 610, the first detection edge 611 or the second detection edge 612 of the detection component 610 may cut through the detection signal. When the first detection edge 611 or the second detection edge 612 is perpendicular to a rotation direction of the rotation component, the detection signal may not be transmitted at the first detection edge 611 or the second detection edge 612, thereby avoiding a generation of an interference signal that is different from the first signal and the second signal. The first signal and the second signal may transit instantaneously when the first detection edge 611 or the second detection edge 612 cuts through the detection signal. This is conducive to improving the accuracy of the detection.

In some embodiments, the detection component 610 may be a fan shaped detection component or any other feasible shape as long as the detection component meets shading and spatial requirements of the sensor 620 during the movement of the transducer unit 230. For example, the fan shaped detection component may correspond to the situation where a line connecting the first detection edge 611 and the second detection edge 612 is a circular arc. For another example, the line connecting the first detection edge 611 and the second detection edge 612 may also be a straight line, and it may be appreciated that a radius angle of the first detection edge 611 and the second detection edge 612 in this situation may not be 180°. For another example, the line connecting the first detection edge 611 and the second detection edge 612 may also be a folded line or other irregularly shaped line. In some embodiments, the radius angle corresponding to the fan shaped detection component may be less than or equal to 180°, such as 30°, 40°, 60°, 90°, 120°, 160°, 180°, etc. The device for transmitting the detection signal of the position detection device 600 (e.g., the sensor 620) to the fan shaped detection component may be perpendicular to a fan shaped surface of the fan shaped detection component. In some embodiments, the fan shaped detection component may correspond to a radius angle equal to 180°, then the fan shaped detection component may also be understood as a semicircular detection component.

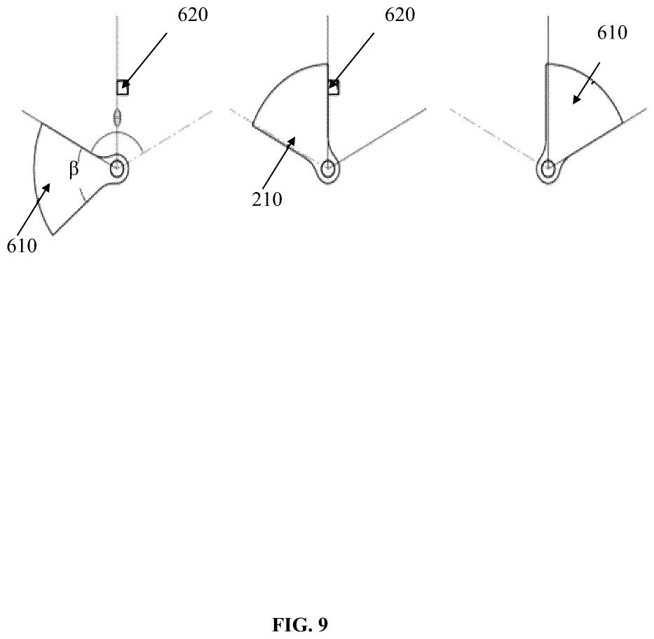

FIG. 9 is a schematic diagram illustrating a structure of a non-semicircular sector shaped detection component according to some embodiments of the present disclosure, and a schematic diagram illustrating a change of relative positions of a sensor and a detection component during a movement of a transducer unit according to some embodiments of the present disclosure. In some embodiments, as shown in FIG. 9, an angle of the detection component 610 may not be 180°, and the angle of the detection component 610 may be greater than or equal to one-half of an oscillation angle of the transducer unit 230. At this time, when the transducer unit 230 performs a single directional movement, the output signal of the sensor 620 may transit, which is conducive to quickly determining an initial position of the transducer unit 230 set. For example, as shown in FIG. 9, during a synchronized movement of the detection component 610 and the transducer unit 230 with an angle β, the relative positional relationship between the detection component 610 and the sensor 620 may further help to implement the detection of the set initial position.

In some embodiments, a material of the detection component 610 may be an aluminum, an aluminum alloy, a magnesium alloy, or an opaque plastic. By choosing the type of lightweight and opaque material, on the one hand, a weight of an ultrasound probe may be reduced so as to improve the ease and comfort of an operation. On the other hand, the detection component 610 may be applied to the implementation of the position detection when the sensor 620 is a photoelectric sensor or an ultrasonic sensor, etc.

In some embodiments, by designing the detection component of the sensor 620 as the fan, an approximate initial position of the transducer unit 230 may be determined by the state of the sensor 620 in an initial state. By controlling the transducer unit 230 to rotate and detect towards a determined direction, the set initial position corresponding to the transducer unit 230 may be quickly found, thereby improving the efficiency of an alignment, and eliminating a risk of a collision with an inner wall of the transducer 200 when the transducer unit 230 is looking for the set initial position.

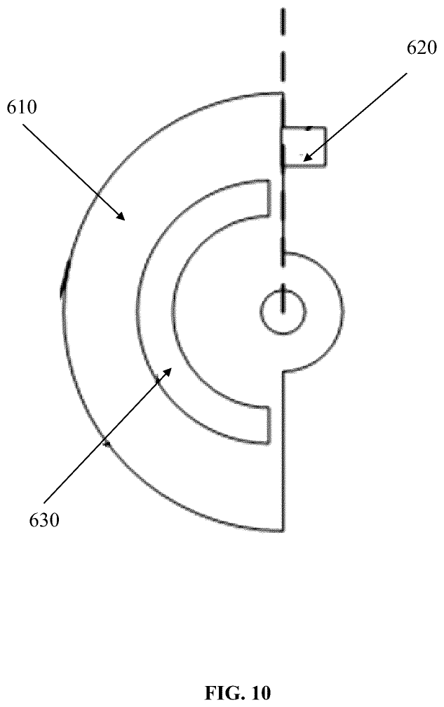

FIG. 10 is a schematic diagram illustrating a detection component with a hollow structure according to some embodiments of the present disclosure. In some embodiments, as shown in FIG. 10, the detection component 610 may also not be entirely solid, and at a position where the detection component 610 and the sensor 620 do not correspond, i.e., the position that do not interfere with a detection function of the sensor 620, there may be a hollow structure 630 to reduce a mass of the detection component 610 and improve a user comfort.

In some embodiments, a plurality of detection regions may be disposed on detection component. The plurality of detection regions may be sequentially arranged along a rotation direction of a rotation component. In some embodiments, during a synchronized movement of the detection component 610 and the rotation component, detection signals may be transmitted sequentially to different detection regions among the plurality of detection regions, and the different detection regions may correspond to different first signals. In some embodiments, the sensor 620 may output a plurality of different first signals, the plurality of different first signals corresponding to the plurality of detection regions, respectively.

In some embodiments, the detection component 610 itself may also act as a sensor to enable the detection of different signals. For example, the detection component 610 may be divided into two types of regions in the rotation direction, namely, region A and region B. Different photoelectric sensing elements may be disposed in the two types of regions (or the same photoelectric sensing elements may be disposed but configured to output different signals), thereby determining the position of the transducer unit 230 through feedback from the sensor.

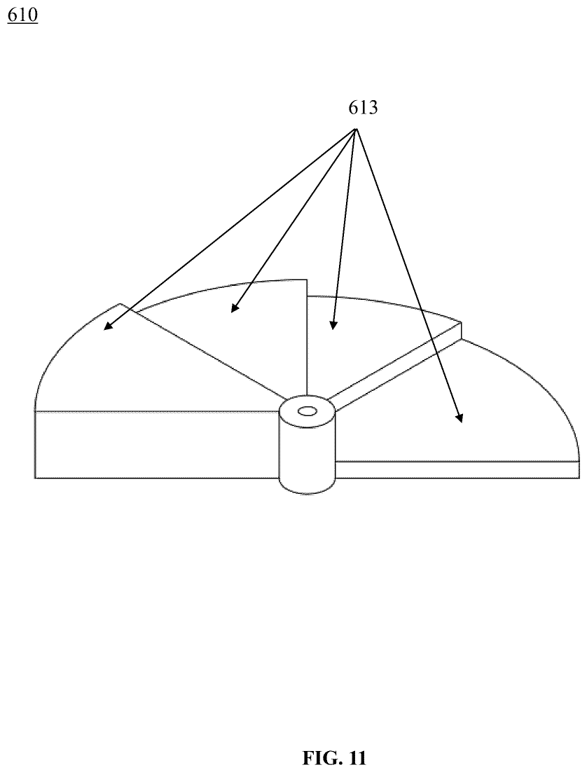

FIG. 11 is a schematic diagram illustrating a structure of a plurality of detection regions according to some embodiments of the present disclosure. In some embodiments, as shown in FIG. 11, the plurality of detection regions may be a plurality of stepped surfaces 613 that are sequentially increasing in height or sequentially decreasing in height along a rotation direction of a rotation component, and during a synchronized movement of the detection component 610 and the rotation component, the detection signal output by the sensor 620 may be transmitted sequentially on the plurality of stepped surfaces 613. The plurality of stepped surfaces 613 may be at different distances from the sensor 620, and thus the signals may be turned on at different levels. As a result, the sensor 620 may output a plurality of different first signals.

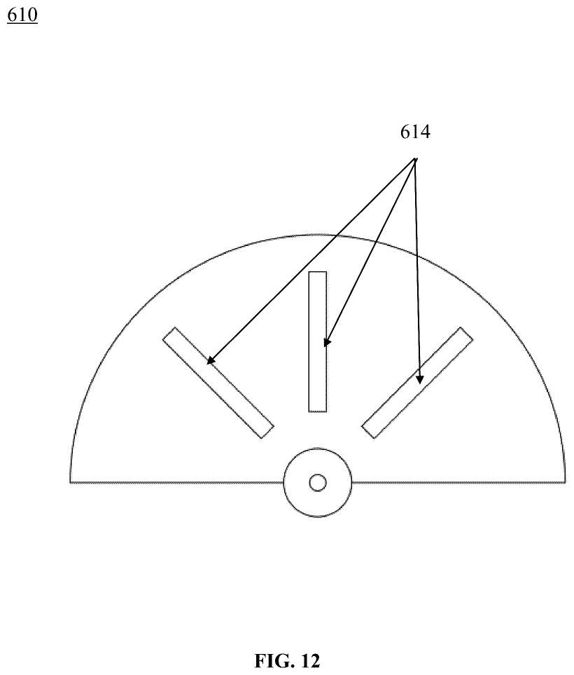

FIG. 12 is a schematic diagram illustrating another structure of a plurality of detection regions according to some embodiments of the present disclosure. In some embodiments, the plurality of detection regions may be regions formed by isolation grooves 614 disposed sequentially in a rotation direction of a rotation component as shown in FIG. 12. During a synchronized movement of the detection component 610 and the rotation component, a detection signal transmitted by the sensor 620 may generate a transitional signal when the detection signal passes the isolation grooves 614. The transitional signal may be different from a first signal. When the transitional signal is generated, a region where the isolation groove 614 passes through an area covered by the detection signal may be determined. During the synchronized movement of the detection component 610 and the rotation component, the sensor 620 may output a plurality of first signals and the transitional signals spaced apart from each other, and the plurality of first signals may be the same signal.

FIG. 13 is a schematic diagram illustrating a structure of a power structure according to some embodiments of the present disclosure.

In some embodiments, as shown in FIG. 13, a rotation component may include the rotation axis 231. The rotation axis 231 may be connected to the transducer unit 230 of the transducer 200. In some embodiments, the transducer unit 230 may be disposed on the transducer base 220, the transducer base 220 may be mounted on a housing of an ultrasound probe. The driven wheel 120 may be connected to the transducer base 220, and the transducer unit 230 may move synchronously with the driven wheel 120 through the transducer base 220. In some embodiments, the detection component 610 may be disposed on the transducer unit 230. In some embodiments, the detection component 610 may be disposed on the rotation axis 231, and a face on which the detection component 610 is disposed may be perpendicular to the rotation axis 231. In some embodiments, the detection component 610 may be fixedly connected to the rotation axis 231, e.g., a vertex of a fan shaped detection component may be fixedly connected to the rotation axis 231. In some embodiments, the vertex of the detection component 610 refers to the vertex of the fan. It may be appreciated that the detection component 610 may rotate with the vertex as a center of rotation during the movement of the detection component 610 with the transducer unit 230. In case the detection component 610 is a semi-circular, it may be understood that the vertex of the detection component 610 is a circle center of the semi-circle.

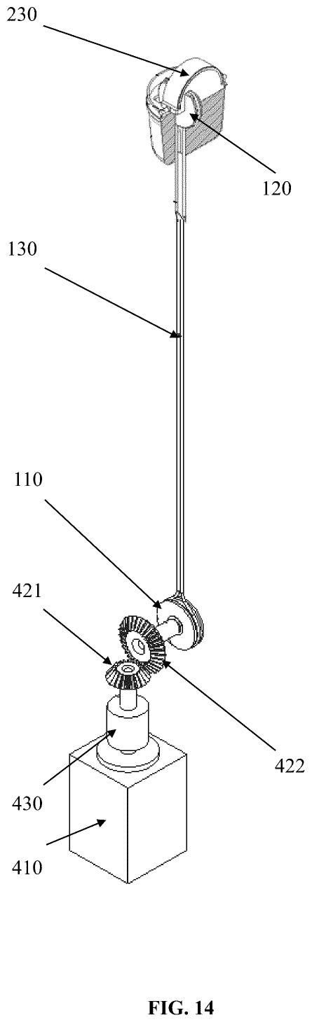

FIG. 14 is a schematic diagram illustrating a structure of a driving assembly according to some embodiments of the present disclosure.

In some embodiments, as shown in FIG. 14, the driving assembly may also include a driving motor 410. An output shaft of the driving motor 410 may be connected to a rotation component. The rotation component may include the driving wheel 110, which may be transmissively connected to the rope 130. The driving wheel 110 may rotate and drive the driven wheel 120 to rotate, so as to drive the transducer unit 230 to move through the transducer base 220.

In some embodiments, the rotation axis 231 may be interference-fitted with the transducer base 220, so that the rotation axis 231 may move synchronously with the transducer base 220. An output shaft of the driving motor 410 may be transmissively connected to the driving wheel 110, and the driving wheel 110 may be transmissively connected to the rope 130. The driving motor 410 may drive the driving wheel 110 to rotate to drive the rope 130 connected to the driving wheel 110 to move. During the moving process, the rope 130 may drive the driven wheel 120 to rotate, so as to drive the transducer base 220 and the rotation axis 231 to rotate, so as to enable the rotation axis 231 to move synchronously with the transducer unit 230. The detection component 610 may be disposed on the rotation axis 231.

In some embodiments, the rotation axis 231 may be interference-fitted with the housing of the ultrasonic probe, and there may be a gap between the rotation axis 231 and the transducer base 220. The output shaft of the driving motor 410 may be transmissively connected to the driving wheel 110, and the driving wheel 110 may be transmissively connected to the rope 130. The driving motor 410 may drive the driving wheel 110 to rotate, so as to drive the rope 130 connected to the driving wheel 110 to move. The movement of the rope 130 may drive the driven wheel 120 to rotate, thereby driving the transducer base 220 to rotate, so that the transducer unit 230 may move. At this time, the rotation axis 231 may not move, and the detection component 610 may be disposed on the transducer unit 230.

In some embodiments, the detection component 610 may be connected to the output shaft of the driving motor 410, and a relative position of the detection component 610 and the output shaft of the driving motor 410 may remain unchanged during an oscillation of the transducer unit 230. In some embodiments, the detection component 610 may be connected to the driving wheel 110 (e.g., the detection component 610 may be disposed on a rotational axis of the driving wheel 110), and the relative position of the detection component 610 and the driving wheel 110 may remain unchanged during the oscillation of the transducer unit 230.

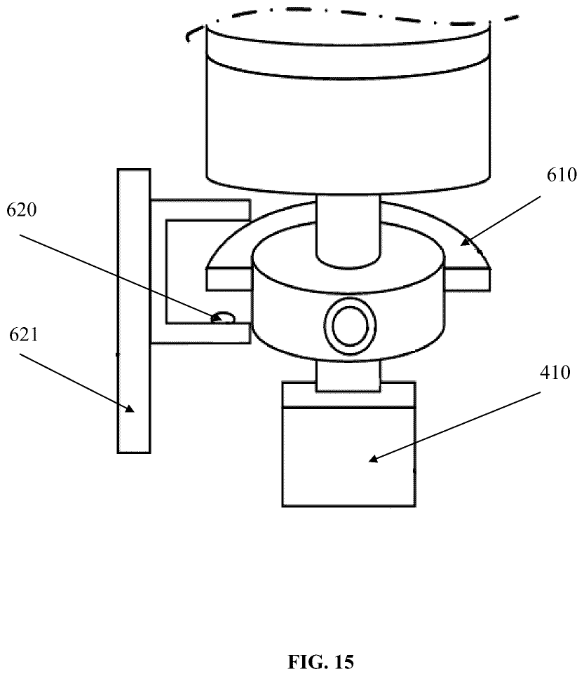

Given that the rotation of the output shaft of the driving motor 410 and the rotational axis of the driving wheel 110 are always synchronized with the transducer unit 230, and rotation angles of these output shafts correspond to the rotation angles of the transducer unit 230, the detection component 610 may be disposed on these output shafts, so that the detection component 610 is able to rotate synchronously with the rotation of the output shafts, and the sensor 620 may be disposed on a substrate that moves relative to these output shafts. For example, the sensor 620 may be disposed on a mounting base 621 of an ultrasonic probe (referring to FIG. 15), and the sensor 620 may not move as the driving assembly drives the transducer unit 230 to rotate. The detection component 610 and the transducer unit 230 may move in synchronization with the movement of the driving assembly, and the relative positions of the sensor 620 and the detection component 610 may change.

In order to further illustrate the change of the relative positions of the detection component 610 to the sensor 620 during the movement of the detection component 610, which causes a change in an output signal of the sensor 620, the following may be illustrated by examples when the sensor 620 is shaded, and the output low level being 0 as well as when the sensor 620 is not shaded, and the output low level being 1.

When the ultrasound probe is activated, the sensor 620 may output one of two states: 0 or 1, depending on the relative position of the detection component 610 and the sensor 620. At a set initial position (e.g., the middle zero position shown in FIG. 7b), the transducer unit 230 may be controlled to rotate clockwise, and if the sensor 620 outputs a low level of 0, it may be determined that the transducer unit 230 is in the state in FIG. 7a, and that the detection component 610 does not shade the sensor 620. The transducer unit 230 may be controlled to continue rotating until the state of the output signal of the sensor 620 changes. Then the position of the output signal change may be determined as the set initial position. As the transducer unit 230 continues to rotate, the relative positional relationship between the sensor 620 and the detection component 610 may be in the state of FIG. 7c, and the output signal of the sensor 620 may remain at 1.

it may be seen from FIG. 8, the set initial position of the transducer unit 230 may be approximately determined through the change of the output signal of the sensor 620, and after the determination, the transducer unit 230 may be controlled to move in one direction to find the set initial position, thereby shortening a time for determining the set initial position. At the same time, the change of the state of the sensor 620 may be an instantaneous point, and based on the point of the state changing, the position of the set initial position (e.g., the intermediate zero position as shown in FIG. 7b) may be accurately determined, thereby improving the accuracy of the alignment.

FIG. 15 is a schematic diagram illustrating a position of a sensor mounted on a mounting base, and a schematic diagram illustrating a detection component mounted on an output shaft of a driving motor according to some embodiments of the present disclosure. In some embodiments, the sensor 620 may be fixedly mounted on a mounting base 621 as shown in FIG. 15. The mounting base 621 refers to a structure that provides a mounting location for the transducer unit 230, for example, the mounting base 621 may be a housing of an ultrasound probe, etc. In some embodiments, the detection component 610 may be detachably connected to an output shaft of the driving motor 410 or, alternatively, the detection component 610 may be detachably connected to a rotational shaft of the driving wheel 110. For example, the detection component 610 may be detachably connected to a connector through an opening hole, the opening hole being a threaded holes, and the connector being a screw. The two may cooperate to realize the detachable connection. The detachable connection may also be implemented by other means such as a fixture. It may be noted that the mounting of the detection component 610 and the sensor 620 may not affect a transmission of the transducer unit 230.

A mounting position of the sensor 620 may depend on the mounting position of the detection component 610 with which it cooperates, and the two should be disposed close to each other, and the sensor 620 may transmit signals to the face where the detection component 610 is located. In order to ensure a detection accuracy, an impact of other mechanical components on the detection result of the position of the detection component 610 on a light path or an ultrasonic transmission path of the sensor 620 for the signal transmission and receiving is avoided. For example, when the position detection of the detection component 610 is performed by utilizing a photoelectric reflection principle of the photoelectric sensor, the light path between the photoelectric sensor and the detection component 610 may be free of other light-shading components, so as to avoid the impact on the detection. The ultrasonic sensor selection may satisfy a similar disposing principle.

In some embodiments, as shown in FIGS. 6b and 7b, when the transducer unit 230 is at the set initial position, the detection signal transmitted by the sensor 620 may be transmitted on a side of the detection component 610 over a vertex. In this position state, regardless of whether the transducer unit 230 is controlled to rotate clockwise or counterclockwise, the output signal of the sensor 620 may jump, and depending on the type of the sensor 620 selected, the output signal of the sensor 620 may change (or jump) from a high level to a low level, or from a low level to a high level. The change (or jump) position may be the set initial position of the transducer unit 230. For example, when it is necessary to determine a middle zero position of the transducer unit 230, i.e., a position shown in FIG. 6b, the relative positions of the sensor 620 and the detection component 610 may be adjusted to where the detection signal transmitted exactly to a side of the detection component passing the vertex when the transducer unit 230 is at the middle zero position. When the ultrasonic probe is subsequently used, the middle zero position may be quickly determined according to the change of the output signal of the sensor 620.

In some embodiments, the sensor 620 may include a photoelectric sensor. The photoelectric sensors may be a device that is capable of converting the light signals into electrical signals. In some embodiments, the photoelectric sensor may be a phototube, a photomultiplier tube, a photoresistor, a photodiode, or a phototransistor.

In some embodiments, the detection signal may be a light signal, and the photoelectric sensor may include a light transmitter and a light receiver. The light transmitter may be configured to transmit the light signal toward the light receiver, and the light receiver may be configured to receive the signal transmitted by the light transmitter. In some embodiments, the light transmitter and the light receiver may be disposed on each side of the detection component 610 along the axis of the rotation axis 231.

In some embodiments, during a synchronized movement of the detection component 610 and a rotation component, the detection component 610 may shade the light signal when passing through a light signal transmission range of the light transmitter, and when the detection component 610 leaves the light signal transmission range of the light transmitter, the light signal may transmit on the light receiver. The light receiver may output a second signal when the light receiver receives the light signal, and may output a first signal when the light receiver does not receive the light signal. In some embodiments, the first signal and the second signal may be signals of opposite levels.

In some embodiments, the light transmitter and the light receiver may be disposed on the same side of the detection component 610. At this time, during the synchronized movement of the detection component 610 and the transducer unit 230, when the light signal is shaded, the light signal may be reflected by the detection component 610 to the light receiver. When the light signal is not shaded, the light signal may not be reflected to the receiver. The light transmitter and the light receiver may also be provided on both sides of the detection component 610, e.g., the detection component 610 may be disposed on the rotation axis 231, the light transmitter may be disposed on the transducer base 220, and the light receiver may be disposed on the housing of the ultrasound probe. The light transmitter and the light receiver may be disposed on both sides of the detection component 610, so that when the detection component 610 shades the light signal, the light receiver may not be able to receive the light signal, and when the detection component 610 does not shade the light signal, the light receiver may receive the light signal transmitted by the light transmitter. A mounting of the photoelectric transducer may be flexibly carried out according to a specific structure of the ultrasonic probe.

In some embodiments, the sensor 620 may include a laser sensor or an ultrasonic sensor.

The ultrasonic transducer may be a transducer that converts an ultrasonic signal into the electrical signal. A change of the electrical signal indicates the change of the ultrasonic signal, and based on the change, a changing situation of the ultrasonic transducer sensing the ultrasound may be obtained. For example, the ultrasonic transducer may transmit ultrasonic waves toward a surface where the detection component 610 is located, and when the ultrasonic waves are transmitted on the detection component 610, they may act on the ultrasonic transducer by reflection. The ultrasonic transducer may output the first signal when receiving the ultrasound signal. When the detection component 610 moves with the movement of the transducer unit 230, the detection component 610 may move from a state of shading the ultrasonic signal to the state of not shading the ultrasonic wave, the ultrasonic sensor may be unable to receive the reflected ultrasonic signal, and the second signal may be output. In some embodiments, the levels of the first signal and the second signal may be different, and based on the difference, there may be a transition position in the electrical signal output by the ultrasonic sensor, which is the transition position corresponding to a set initial position of the transducer unit 230. In this way, it is not necessary to open the housing of the transducer unit 230 to know that the transducer unit 230 is currently at the set initial position.

In some embodiments, the laser sensor may be a light transmitter for transmitting a single beam of light. When the light transmitter is transmitted as the single beam of light, a single-point detection may be performed, and the transition of the sensor 620 may be clearer, so as to further improve the accuracy of the detected set initial position.

In some embodiments, during the oscillation of the transducer unit 230, the movement of the detection component 610 may be able to synchronize and reflect the oscillation of the transducer unit 230, and then, by disposing the sensor 620, the oscillation of the transducer unit 230 may be indicated, so as to determine the position of the transducer unit 230. During the synchronized oscillation of the detection component 610 and the transducer unit 230, at a moment when a side of the detection component 610 cuts through the sensor 620, the output signal of the sensor 620 may transit. Based on the feature, the position of the transducer unit 230 may be accurately and quickly determined, thereby optimizing a problem of deviation of the corresponding position of the ultrasound probe during a forward and reverse scanning. Additionally, when the ultrasound probe is turned on, the high and low levels of the output signals from the sensor 620 may be used to quickly determine an approximate orientation of the transducer unit 230, and then determine whether the transducer unit 230 should be rotated clockwise or counterclockwise to quickly find the set initial position, so as to avoid a collision noise when turning in a wrong direction.

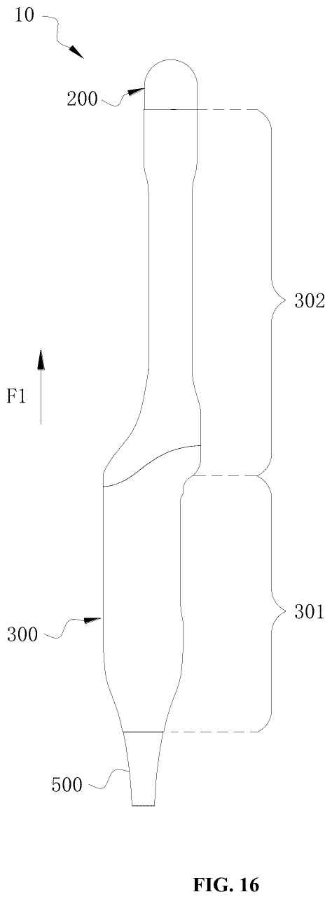

FIG. 16 is a schematic diagram illustrating a structure of an ultrasonic probe according to some embodiments of the present disclosure. In some embodiments, as shown in FIG. 16, an ultrasound probe 10 may include the power structure of the embodiments described above and a transducer 200. The transducer 200 may be configured to transmit and receive an ultrasound signal, and the transducer 200 may be connected to the driven wheel 120 of the power structure.

It may be understood that the ultrasound probe 10 provided in the embodiments of the present disclosure may not only be used in the body, but also be used in a medical scenario such as a body surface. A selection may be made based on an actual use.

The following is an example of the ultrasound probe used in the body, and the ultrasound probe 10 is described in conjunction with the accompanying drawings.

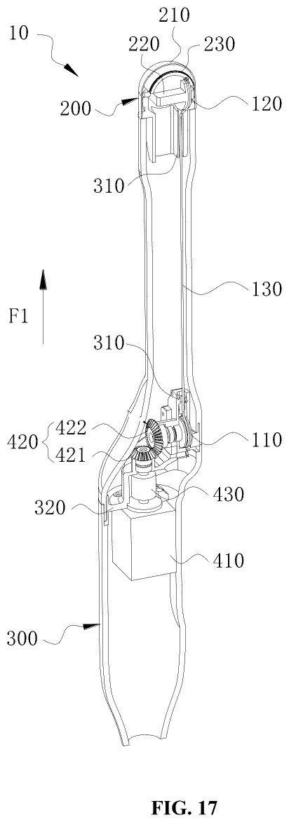

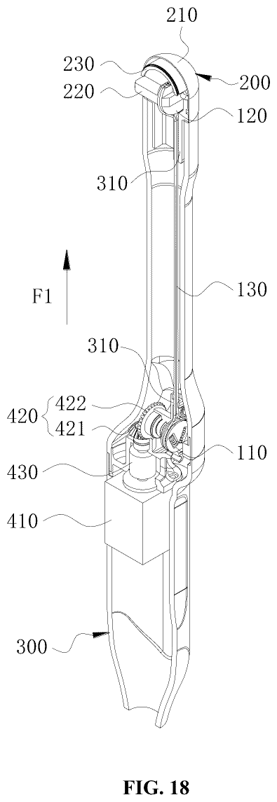

FIG. 17 is a schematic diagram illustrating a portion of structure of an ultrasonic probe in one perspective according to some embodiments of the present disclosure. FIG. 18 is a schematic diagram illustrating a portion of structure of an ultrasonic probe in another perspective according to some embodiments of the present disclosure.

In some embodiments, as shown in FIGS. 16, 17, and 18, the ultrasound probe 10 may further include a housing 300. The transducer 200 may be disposed at one end of a longitudinal direction (a first direction F1 illustrated in the figure) of the housing 300. A power structure may be disposed within the housing 300, and a rotation component of the power structure may include a driving wheel 110, the driving wheel 110 being transmissively connected to the driven wheel 120, the driven wheel 120 being connected to the transducer 200.

The housing 300 may be a component for accommodating the power structure, a driving assembly, and a cable connecting the transducer 200. In some embodiments, the housing 300 may generally be a longitudinally elongated component extending along the first direction F1. Along the first direction F1, the housing 300 may have a grip section 301 and an intrusion section 302 sequentially connected. The grip section 301 may be provided for an operator to grip, and the intrusion section 302 may be provided for reaching into a position to be detected in a human body. In some embodiments, the grip section 301 and the intrusion section 302 may be of an integrated structure or a split structure. In some embodiments, when the grip section 301 and the intrusion section 302 are of the split structure, the grip section 301 and the intrusion section 302 may be connected and fixed by means of bonding, which is more convenient for manufacturing and installation.

The transducer 200 may be disposed at one end of the intrusion section 302 away from the grip section 301, one end of a connection cable 500 may extend from one end of the grip section 301 away from the intrusion end 302 into the housing 300, and may be connected to the transducer 200. The other end of the connection cable 500 may be electrically connected to a body of the ultrasound device. The ultrasound signal transmitted and received by the transducer 200 may be transmitted to the body of the ultrasound device with the help of the connection cable 500 to obtain a 3D image or a 4D image of a human tissue. The transducer 200 may also be attached and fixed to one end of the intrusion section 302 away from the grip section 301 by bonding.

The power structure may transmit a driving force to the transducer 200 to enable a rotation of the transducer unit 230 within the transducer 200. The power structure may include the rope 130 through which the driving force of the driving motor 410 may be transmitted from the driving wheel 110 to the driven wheel 120. The transmission of the rope 130 may have a good flexure, which makes the transmission process smoother and avoids a noise, a vibration, and an impact. For the transducer 200, a flexible bending may be performed to avoid damaging the transducer 200 during use.

In some embodiments, the driven wheel 120 and the transducer 200 may be disposed on the same side, i.e., near the end of the intrusion section 302 away from the grip section 301, while as the intrusion section 302 is required to be reached inside the body, and a size of the intrusion section 302 may be inevitably limited due to a space constraint. Therefore, in order to utilize the space even further, the elastic component 140 may be disposed within the driving wheel 110 as in some of the aforementioned embodiments. As the driving wheel 110 is disposed in the grip section 301, the grip section 301 may be disposed outside of the body, and may have a greater use of space, which is conducive to increasing a transmission ratio of the transmission device 100. The other ends of the two ropes 130 may be fixed to opposite sides of the driven wheel 120 with the help of connectors such as screws for a fixed connection.

In some embodiments, the housing 300 may be provided with a limit structure 310. The limit structure 310 may be configured to gather the rope 130 disposed between the driving wheel 110 and the driven wheel 120. That is, in conjunction with reference to FIGS. 10 and 11 illustrated hereinafter, it is possible to gather the rope 130 disposed between driving wheel 110 and the driven wheel 120 together, so as to reduce a space within the housing 300 occupied by the transmission 100.

In some embodiments, the limit structures 310 may be disposed in the housing 300 near the positions of the driving wheel 110 and the driven wheel 120, and a restriction channel may be formed in the limit structure 310. The two ropes 130 may be close to each other through the limit channel and may be limited in the limit channel. The restriction structure 310 may be integrated with the housing 300, or it may be a structure split form the housing 300, as long as a gathering effect is able to be realized. In some embodiments, the limiting structure 310 may also be disposed at a position within the housing 300 near the driven wheel 120.

It should be noted that the limiting structure 310 illustrated in FIGS. 17 and 18 may be formed on a corresponding base within the housing 300 for mounting the transducer 200 and the driving assembly. In some embodiments, the limiting structure 310 may also be provided at other positions within the housing 300 as long as a limiting effect is able to be achieved.

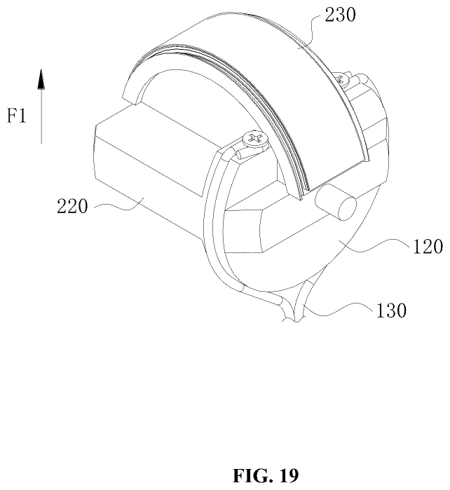

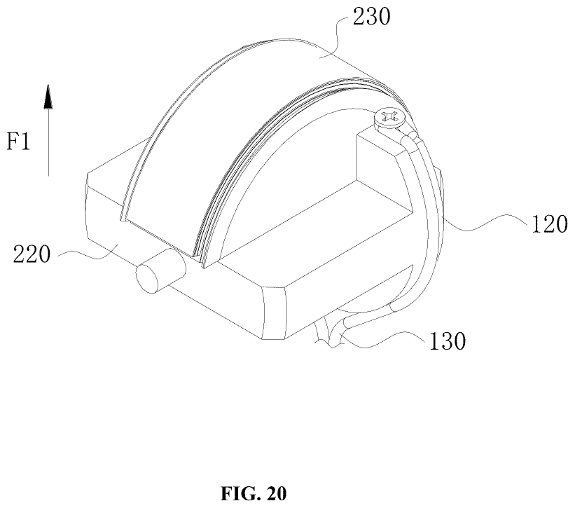

FIG. 19 is a schematic diagram illustrating a structure of a transducer cooperating with a driving wheel in one perspective according to some embodiments of the present disclosure. FIG. 20 is a schematic diagram illustrating a structure of a transducer cooperating with a driven wheel in another perspective according to some embodiments of the present disclosure.

In some embodiments, as shown in FIGS. 17-20, the transducer 200 may include a housing 210, the transducer base 220 disposed within the housing 210, and the transducer unit 230 disposed on the transducer base 220. The transducer base 220 may be fixedly connected to one end of the intrusion section 302 of the housing 300, or may be integrated with the intrusion section 302 of the housing 300. The transducer unit 230 may be fixed to the transducer base 220, and the transducer unit 230 may be integrally and rotatably connected to the limit structure 310 by the rotation axis 231. The transducer unit 230 may oscillate relative to the limit structure 310. The transducer 200 may be fixedly connected to the housing 100 with a profile limit of the limit structure 310, and the housing 210 may be connected to the limit structure 310 and sealed by a sealing structure. In some embodiments, the transducer base 220 and the driven wheel 120 may be of an integrated structure or split structures. In the situation where the transducer base 220 and the driven wheel 120 are integrated, the limit structure 310 may be similar to a base body disposed on the housing 300 for mounting the transducer 200.

In some embodiments, a rolling bearing may be disposed within the limit structure 310 to change a sliding friction between the rope 130 and the limit structure 310 into a rolling friction, thereby reducing the friction and avoiding aware of the rope 130.

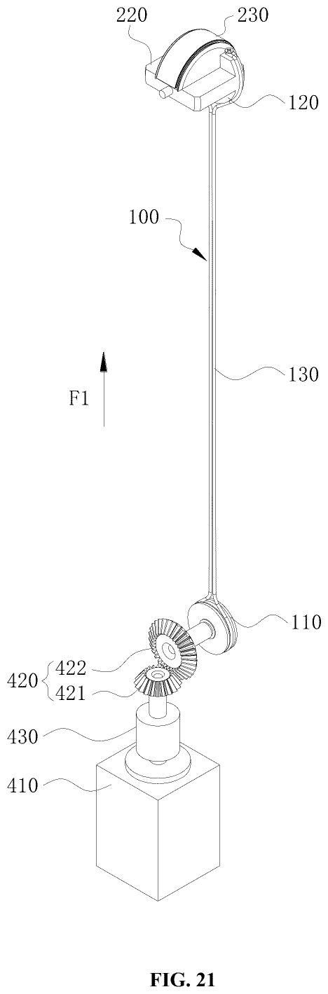

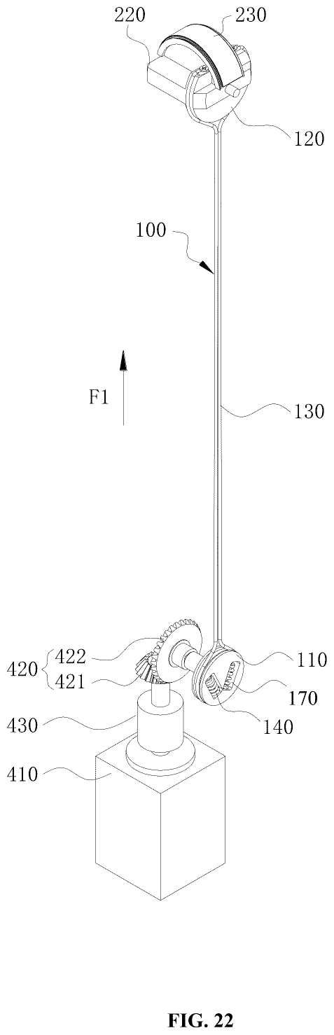

FIG. 21 is a schematic diagram illustrating a structure of a power structure in one perspective according to some embodiments of the present disclosure. FIG. 22 is a schematic diagram illustrating a structure of a power structure in another perspective according to some embodiments of the present disclosure.

In some embodiments, as shown in FIGS. 21 and 22, the driving motor 410 may be transmissively connected to a gear set 420. An output end of the gear set 420 may be transmissively connected to the driving wheel 110. A driving of the power structure may be realized by disposing the driving motor 410 and the gear set 420.

In some embodiments, as shown in FIGS. 21 and 22, the gear set 420 may include a first bevel gear 421 that is transmissively connected to the driving motor 410, and a second bevel gear 422 that is engaged with the first bevel gear 421. The second bevel gear 422 may be connected to the driving wheel 110 through a transmission shaft to drive the driving wheel 110 to rotate around an axis of the transmission shaft. In some embodiments, the driving motor 410 and the first bevel gear 421 may be connected to each other through a coupling 430. When the driving motor 410 rotates and drive the first bevel gear 421 to rotate, the coupling 430 may correct an eccentricity of an output shaft of the driving motor 410 and a gear shaft of the first bevel gear 421. By disposing the first bevel gear 421 and the second bevel gear 422, without taking up too much space and with a more compact structure, the transmission of the power may be smoother.

In some embodiments, connection ends of the rotational shaft of the second bevel gear 422 and the rotational shaft of the driving wheel 110 may be disposed as a square shaft and a square hole, respectively, and may be fastened by screws. When the second bevel gear 422 rotates, it may drive the driving wheel 110 to rotate synchronously. The transmission process between the second bevel gear 422 and the driving wheel 110 may be made more stable through a cooperation of the square shaft and the square hole.

In some embodiments, an oscillation of the transducer 200 may be achieved by forward and reverse rotation of the driving motor 410. In some embodiments, the driving motor 410 may also be replaced by another device capable of generating a driving force, such as a rotary cylinder, etc., which can be set according to an actual space to be used, and the embodiments of the present disclosure do not specifically limit this.

In some embodiments, as shown in FIGS. 17 and 18, the driving motor 410 may be arranged at a connection of the grip section 301 and the intrusion section 302, thereby avoiding a use of an excessively long output shaft in the driving motor 410 and preventing a deformation thereof. In some embodiments, a base 320 may be provided at the grip section 301 and the intrusion section 302. The base 320 may be connected to an inner wall of the housing 300. The driving motor 410 may be mounted to the base 320. In some embodiments, the second bevel gear 422 and the driving wheel 110 may be fixed in the base 320 by the roller bearing. Thus, while avoiding compromising the arrangement due to space constraints in the intrusion section 302, the space in the grip section 301 may be fully utilized.

In some embodiments, the limit structure 310 may be disposed on the base 320 to gather the rope 130 proximate to the driving wheel 110.

In some embodiments, a sectional area of the intrusion section 302 may be smaller than the section area of the grip section 301 along the longitudinal direction, as shown in FIGS. 16, 17, and 18. The grip section 301 may be greater in volume for an operator to grip, while the intrusion section 302 may be smaller for extending into the human body for scanning and imaging of the transducer 200.

In some embodiments, an on/off button of the driving motor 410 may be disposed in the grip section 301 for ease of use by the operator. For example, the driving motor 410 may be set to be able to rotate forward and reverse. The switch button may be set according to actual use requirements, and the embodiments of the present disclosure do not make specific limitations thereon.

In application, a physician may grip the grip section 301 of the housing 300, extend the intrusion section 302 into a hoped detection position, and control the movement of the transducer unit 230 inside the transducer 200 through the buttons of the grip section 301 to realize the scanning and imaging of the probe. The specific transmission process may be that the first bevel gear 421 is driven to rotate when the driving motor 410 rotates, which in turn drives the second bevel gear 422 that meshes with the first bevel gear 421 to rotate. When the second bevel gear 422 rotates, the second bevel gear 422 may drive the driving wheel 110 to rotate synchronously. The driving wheel 110 may drive the driven wheel 120 to rotate through the rope 130, which in turn drives the transducer unit 230 to rotate.

In some embodiments, by arranging the driving motor 410 at a connection between the intrusion section 302 and the grip section 301, and using the driving motor 410 to drive the transducer 200. In this way, the transmission may be smoother and more reliable, and as the transducer 200 may bend flexibly, the transducer 200 may not be damaged. Furthermore, as a gear transmission is used in a primary transmission, the transmission may be smooth and accurate, the structure may be compact without taking up any volume, and the rope is used in a secondary transmission, which enables a good flexibility and a smooth transmission, and a noise, a vibration, and an impact is avoided. Furthermore, by providing the flexible component 140, while ensuring a tension of the rope 130, the impact during the starting and stopping process may be reduced, so as to make the transmission process smoother. By disposing the elastic component 140 inside the body of the wheel of the driving wheel 110 or the driven wheel 120, no additional internal space of the device may be taken, which further makes the structure more compact and more reliable. By using the guiding component 150 based on the elastic component 140, a bending of the elastic component 140 is avoided, so as to improve the accuracy and reliability of the transmission.

In some embodiments, the ultrasound probe may further include the position detection device 600, the position detection device 600 including the detection component 610. The transducer 200 may include the transducer unit 230, and the power structure of the foregoing embodiment may drive the transducer unit 230 to oscillate, and the detection component 610 may move synchronously with the transducer unit 230, as may be described in the preceding descriptions.

In some embodiments, through the power structure, the position of the transducer unit 230 may be quickly determined without the need for ultrasonic probe disassembly, which provides an important data basis for the control of the scanning imaging of the transducer unit 230, and the initial position set by the transducer unit 230 may be quickly and accurately determined. In the process of reciprocating oscillation of the transducer unit 230, a set initial position detection may be performed during each single direction movement process, which is more conducive to further eliminate the impact of a cumulative error in the scanning process of the transducer unit 230, so as to improve the quality of imaging.

Some embodiments of the present disclosure further provide an ultrasound imaging system, which may include the ultrasound probe of the above embodiments, a host, and a display. The ultrasound probe may be configured to transmit and/or receive the ultrasound signal. The host may include one or more processors, and the host may be configured to perform calculations on the ultrasound signals and determine medical images. The display may be electrically connected to the host, and the display may be configured to display the medical images.

In some embodiments, in the reciprocating movement of the transducer unit 230, the position detection device 600 of the ultrasound imaging system may perform a plurality of positionings based on the set initial position, so as to provide the data basis for the ultrasound imaging, thereby ensuring the imaging quality.

The basic concepts have been described above, and it is apparent to those skilled in the art that the foregoing detailed disclosure is intended as an example only and does not constitute a limitation of the present disclosure. While not expressly stated herein, various modifications, improvements, and amendments may be made to the present disclosure by those skilled in the art. These modifications, improvements, and amendments are suggested in the present disclosure, so these modifications, improvements, and amendments remain within the spirit and scope of the exemplary embodiments of the present disclosure.

Also, the present disclosure uses specific words to describe embodiments of the present disclosure. such as “an embodiment”, “one embodiment”, and/or “some embodiment” means a feature, structure, or characteristic associated with at least one embodiment of the present disclosure. Accordingly, it should be emphasized and noted that “one embodiment” or “an embodiment” or “an alternative embodiment” referred to two or more times in different locations in the present disclosure may not refer to the same embodiment. In addition, certain features, structures, or characteristics in one or more embodiments of the present disclosure may be suitably combined.

Similarly, it should be noted that in order to simplify the expression of the present disclosure, and thereby helps in the understanding of one or more embodiments of the present disclosure, the foregoing descriptions of embodiments of the present disclosure sometimes group multiple features together in a single embodiment, accompanying drawing, or the description thereof. However, this mode of disclosure does not imply that the objects of the present disclosure require more features than those mentioned in the claims. Rather, the claimed subject matter may lie in less than all features of a single foregoing disclosed embodiment.

Some embodiments use numbers to describe the number of components, attributes, and it should be understood that such numbers used in the description of embodiments are modified in some examples by the modifiers “approximately”, “nearly”, or “substantially”. Unless otherwise noted, the terms “approximately”, “nearly”, or “substantially” indicates that a ±20% variation in the stated number is allowed. Correspondingly, in some embodiments, the numerical parameters used in the present disclosure and claims are approximations, which approximations are subject to change depending on the desired characteristics of individual embodiments. In some embodiments, the numerical parameters should consider the specified number of valid digits and use a general digit retention method. While the numerical domains and parameters used to confirm the breadth of their ranges in some embodiments of the present disclosure are approximations, in specific embodiments such values are set to be as precise as possible within a feasible range.

For each patent, patent application, patent application disclosure, and other material cited in the present disclosure, such as articles, books, specification sheets, publications, documents, etc., the entire contents of which are hereby incorporated herein by reference. Except for application history documents that are inconsistent with or create a conflict with the contents of the present disclosure, and except for documents that limit the broadest scope of the claims of the present disclosure (currently or hereafter appended to the present disclosure). It should be noted that in the event of any inconsistency or conflict between the descriptions, definitions, and/or use of terminology in the materials appended to the present disclosure and those set forth in the present disclosure, the descriptions, definitions and/or use of terminology in the present disclosure shall prevail.

Finally, it should be understood that the embodiments described in the present disclosure are used only to illustrate the principles of the embodiments of the present disclosure. Other deformations may also fall within the scope of the present disclosure. Therefore, alternative configurations of embodiments of the present disclosure may be viewed as consistent with the teachings of the present disclosure as an example, not as a limitation. Correspondingly, the embodiments of the present disclosure are not limited to the embodiments expressly presented and described herein.

What is claimed is:

1. A power structure, including a driving assembly, wherein the driving assembly includes a transmission device, the transmission device including a rotation component, a rope and an elastic component, the rotation component including a driving wheel and/or a driven wheel; wherein
the driving wheel is in a transmission connection to the driven wheel by the rope;
the rope is in an elastic connection to a body of the driving wheel and/or a body of the driven wheel through the elastic component;
the rotation component is provided with an accommodation cavity, and an extension channel in communication with the accommodation cavity; and
the elastic component is accommodated in the accommodation cavity; one end of the rope elastically connected to the driving wheel and/or the driven wheel is located in the accommodation cavity, and a portion of the rope is located in the extension channel.

2. The power structure of claim 1, wherein an elasticity direction of the elastic component is parallel to an extension direction of the extension channel.

3. The power structure of claim 1, wherein an axis of the elastic component is collinear with an axis of the extension channel.

4. The power structure of claim 1, wherein the extension channel includes a linear extension section; and a ratio of a length of the linear extension section to a diameter of the rope within the linear extension section is greater than 1.5.

5. The power structure of claim 1, wherein
the transmission device includes two elastic components;
two accommodation cavities are arranged within the rotation component, and the two elastic components are disposed in the two accommodation cavities, respectively;
the two elastic components are arranged on both sides of a radial reference plane of the rotation component; and
a rotation axis of the rotation component is located on the radial reference plane.

6. The power structure of claim 5, wherein
the two elastic components are symmetrically arranged on the both sides of the radial reference plane, and the extension directions of the extension channels corresponding to the two elastic components are disposed at an angle;
for each of the extension channels corresponding to the two elastic components, the extension channel has a first end connected to the corresponding accommodation cavity and a second end away from the corresponding accommodation cavity;
a distance between the first ends of the two extension channels is greater than a distance between the second ends of the two extension channels.

7. The power structure of claim 6, wherein the second ends of the two extension channels are in communication with each other.

8. The power structure of claim 7, wherein a separation portion is formed between the two extension channels;
the separation portion includes a first arc section, the first arc section being disposed on an end of the separation portion towards a connection position of the second ends of the two extension channels.

9. The power structure of claim 6, wherein
a second arc section is disposed between the second end of the extension channel and a circumferential edge of the body of the driving wheel or the driven wheel.

10. The power structure of claim 9, wherein the two second arc sections are symmetrically arranged on both sides of the radial reference plane.

11. The power structure of claim 1, further comprising a guiding component disposed within the accommodation cavity; wherein
the guiding component is configured to guide a movement of the elastic component along an elasticity direction of the elastic component.

12. The power structure of claim 1, further comprising a position detection device; wherein the position detection device includes:
a detection component disposed on the rotation component, and the detection component being configured to be driven by the rotation component to perform a synchronized movement with the rotation component; and
a sensor configured to receive a detection signal, and the detection signal being transmitted toward the detection component; wherein
during the synchronized movement of the detection component with the rotation component, a first signal is output by the sensor when the detection signal is transmitted to the detection component, and a second signal is output by the sensor when the detection signal is not transmitted to the detection component; and
the first signal is different from the second signal.

13. The power structure of claim 12, wherein the detection component is a fan-shaped detection component, and the detection signal transmitted by the position detection device to the fan-shaped detection component is perpendicular to a fan-shaped surface of the fan-shaped detection component.

14. The power structure of claim 12, wherein
a plurality of detection regions are arranged on the detection portion, the plurality of detection regions being sequentially disposed along a rotation direction of the rotation component; and
during the synchronized movement of the detection component with the rotation component, the detection signals are emitted sequentially onto different detection regions of the plurality of detection regions, different detection regions corresponding to different first signals respectively.

15. The power structure of claim 12, wherein
the rotation component includes a rotation axis, the rotation axis being connected to a transducer; and
the detection component is disposed on the rotation axis, the detection component being located on a surface perpendicular to the rotation axis.

16. The power structure of claim 15, wherein the driving assembly further includes a driving motor, an output shaft of the driving motor being connected to the rotation component, the rotation component including the driving wheel, the driving wheel being in transmission connection to the rope, the driven wheel being configured to be driven by a rotation of the driving wheel to rotate so as to drive a transducer unit of the transducer to move; and
the detection component is connected to the output shaft of the driving motor, and during an oscillation of the transducer unit, a relative position between the detection component and the output shaft of the driving motor is kept unchanged; or, the detection component is connected to the driving wheel, and during the oscillation of the transducer unit, a relative position between the detection component and the driving wheel is kept unchanged.

17. The power structure of claim 12, wherein
the sensor includes a photoelectric sensor;
the detection signal is a light signal, the photoelectric sensor including a light transmitter and a light receiver;
the light transmitter and the light receiver are respectively disposed on both sides of an axial direction of the rotation axis, and the light transmitter is used for transmitting the light signal towards the light receiver;
during the synchronized movement of the detection component with the rotation component, the detection component blocks the light signal when passing a light signal transmission range of the light transmitter, and the light signal is transmitted to the light receiver when the detection component leaves the light signal transmission range of the light transmitter;
the second signal is output when the light receiver receives the light signal, and the first signal is output when the light receiver does not receive the light signal; and
the first signal and the second signal are signals of opposite levels.

18. The power structure of claim 12, wherein the sensor includes a laser sensor or an ultrasonic sensor.

19. An ultrasound probe, comprising a transducer and the power structure of claim 1, the transducer being used for transmitting and receiving an ultrasound signal, and the transducer being coupled to a driven wheel of the power structure.

20. The ultrasound probe of claim 19, further including a position detection device, the position detection device including a detection component, the transducer including a transducer unit, the power structure being configured to drive the transducer to move; and the detection component being configured to move synchronously with the transducer unit.
